(19) United States
(12) Patent Application Publication
(10) Pub. No.: US 2007/0207123 A1
(43) Pub. Date: Sep. 6, 2007

(54) Amelioration of effects of cigarette smoke

(76) Inventors: Mark L. Witten, Tucson, AZ (US); David T. Harris, Tucson, AZ (US)

Correspondence Address: BANNER & WITCOFF, LTD., 1100 13th STREET, N.W., SUITE 1200, WASHINGTON, DC, 20005-4051, US

(21) Appl. No.: 10/645,839

(22) Filed: Aug. 22, 2003

Related U.S. Application Data
(60) Provisional application No. 60/406,036, filed on Aug. 27, 2002.

(51) Int. Cl. A61K 48/00; A61K 38/17; A61K 9/12

(52) U.S. Cl. 424/93.2; 424/45; 514/12; 514/44

(57) ABSTRACT

Aerosolized Substance P can be used to mitigate the effects of main-stream or side-stream cigarette smoke. Functional, structural, genetic, organ limited, and systemic effects of the smoke are mitigated by the Substance P treatment.

This application claims the benefit of provisional application Ser. No. 60/406,036 filed Aug. 27, 2002, the contents of which are expressly incorporated herein.

### FIELD OF THE INVENTION

The invention relates to the fields of cancer and lung disease. In particular it relates to such diseases caused by cigarette smoke exposure.

### BACKGROUND OF INVENTION

Environmental toxicants may have significant effects on many physiological systems of the exposed individual. For example, significant changes in immune competence in the lung, even if short-lived, may have serious consequences for the exposed host that may affect susceptibility to infectious agents, particularly if combined with pulmonary cellular damage. Major alterations in lung and immune function that are long lasting may result in an increased likelihood of development and/or progression of cancer and other pathological states. Cigarette smoke, whether first-hand or second-hand (i.e., bystander, side-stream, SSCS) is one such environmental toxicant. Both first-hand and second-hand exposure to cigarette smoke is known to damage the lungs, suppress the immune system, and predispose individuals to the development of lung cancer and emphysema (1).

Short-term (7 days) exposure of C57B16 mice to low concentrations of environmental hydrocarbons (i.e., jet fuels) results in profound and significant alterations in the pulmonary and immune systems (2-12). Moreover, hydrocarbon exposure results in a depletion of substance P in the bronchoalveolar fluids of the lung (13). Substance P is a molecule implicated in airway reactivity (14) and pulmonary epithelial cell integrity (15).

Substance P (SP) is a naturally occurring small, molecular weight peptide (11 amino acids) that is localized to the nerves in the airways of several species, including humans (16, 17). Substance P preferentially activates NK-1 tachykinin receptors (18). When SP is administered in vivo by infusion or inhalation it does not induce bronchoconstriction, in contrast to other tachykinins (19). In experiments in which endogenous lung SP was depleted by capsaicin injection (9), the effects of hydrocarbon exposure on the pulmonary system were observed to be more severe. Significantly, aerosolized SP administered to jet fuel exposed animals reversed and/or prevented many of the resulting pathological lung effects (20) and the immunotoxicological effects (4, 7) of the jet fuel exposure. Administration of a concentration as low as 1 uM SP for as shorta time as 15 minutes after hydrocarbon exposure was sufficient to protect exposed animals.

Cigarette smoke poses a health risk to both smokers and non-smokers alike. Side-stream smoke, as experienced by those in smoky environments such as bars and doorways of public buildings, causes a deterioration of lung function and structure, and can lead to genetic changes, which are the precursors to cancer.

There is a need in the art for preventive and therapeutic treatments for ameliorating the effects of cigarette smoke on the human body.

### BRIEF SUMMARY OF THE INVENTION

In one embodiment of the invention a method is provided for ameliorating or preventing damage caused by cigarette smoke. Substance P or a bioactive analog thereof is administered via aerosol inhalation to a subject who has or will be exposed to cigarette smoke. The bioactive analog is selected from the group consisting of [Met-OH11]-substance P, [Met-OMe11]-substance P, [Nle11]-substance P, [Pro9]-substance P, [Sar9]-substance P, [Tyr8]-substance P, [p-Cl-Phe7,8]-substance P, and [Sar9,Met (02)11]-substance P.

In a second embodiment of the invention a method is provided for ameliorating or preventing damage caused by cigarette smoke wherein substance P or a bioactive analog thereof is administered via an attached or attachable filter to a cigarette.

In a third embodiment of the invention a method is provided for ameliorating or preventing damage caused by cigarette smoke wherein substance P or a bioactive analog thereof is administered via a gum or lozenge.

A fourth embodiment of the invention is a cigarette filter comprising substance P or a bioactive analog.

A fifth embodiment of the invention is a gum or lozenge comprising substance P or a bioactive analog.

In a sixth embodiment of the invention a method is provided for ameliorating or preventing damage caused by cigarette smoke wherein a polynucleotide encoding a secretable substance P protein or bioactive analog is administered via a polynucleotide to a human or an animal.

### BRIEF DESCRIPTION OF THE DRAWINGS

FIG. 1. Electron micrograph (˜8,000 magnification) of cigarette smoke-exposed airway. First arrow indicates loss of airway cilia. Double arrows indicate swelling in airway basement membranes. Cigarette smoke exposure consisted of 45 min/day sidestream cigarette smoke for 14 consecutive days.

FIG. 2. Electron micrograph (˜8,000 magnification) of cigarette smoke-exposed airway. Single arrow indicates normal cilia. Double arrow indicates normal appearance of airway basement membranes. Cigarette smoke exposure consisted of 45 min/day sidestream cigarette smoke followed by a 15 min aerosol dose of Sar⁹, Met (O₂)¹¹-substance P for 14 consecutive days.

FIG. 3. Electron micrograph (˜8,000 magnification) of cigarette smoke-exposed airway. Single arrow indicates identifiable cell membrane between airway cells. Cigarette smoke exposure consisted of 45 min/day sidestream cigarette smoke followed by a 15 min aerosol dose of Sar⁹, Met (O₂)¹¹-substance P for 14 consecutive days.

FIG. 4 Electron micrograph (˜8,000 magnification) of cigarette smoke exposed airway. Arrows indicate cell swelling and no easily identifiable cell membranes between airway cells. Cigarette smoke exposure consisted of 45 min/day sidestream cigarette smoke for 14 consecutive days.

### DETAILED DESCRIPTION OF THE INVENTION

It is a discovery of the present inventors that aerosol administration of substance P or a bioactive analog thereof can ameliorate or prevent various types of damage wreaked by cigarette smoke. The smoke can be main-stream or side-stream. Types of damage for which a positive effect of Substance P have been observed include dynamic lung compliance, basement membrane structure of endothelial cells of airways, and micronuclei formation.

Aerosolization has been found to be a very effective means of administering Substance P to mammalian subjects. However, other means, as are known in the art, such as intravenous, subcutaneous, intramuscular, intraperitoneal, and intraarterial administration can be used as alternatives. Typically these will be by injection, although other delivery means can also be used such as transdermal absorption. In addition delivery may be effectuated via a filter of a cigarette, cigar, pipe, or other smoking product, or a gum or lozenge. Any such means as is known in the art can be applied.

Substance P (RPKPQQFFGLM-NH₂) (SEQ ID NO:1) or any of its bioactive analogues can be used in the methods of the present invention. These include, but are not limited to: [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met(0₂)¹¹]-substance P. The latter analogue is particularly preferred. Bioactive analogs, according to the invention are those which act as competitive inhibitors of SP by binding to the SP receptor (NK-1 receptor). Other derivatives as are known in the art and commercially available (e.g., from Sigma) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same amino acid backbone. Again, routine testing will determine which of such modifications do not adversely affect biological activity.

Typical concentration ranges of substance P or its bioactive analogue in the aerosol administered is between 0.001 and 10 μM. Concentrations in the range of between 0.05 and 5 μM are particularly useful. It can be advantageously administered as a liquid at a concentration between about 0.1 and 10 μM. It may be administered via the filter of a cigarette at a concentration between about 0.1 and 10 μM. It may also be administered at a concentration between about 0.1 and 10 μM via a gum or lozenge.

Suitable devices for administering the aerosol of the present invention include nebulizers as well as hand-held aerosol “puffer” devices. Filters can also be used, as discussed above. Filters can be made according to any method known in the art. Natural or synthetic fibers can be used in the filters, for example. The filters can be impregnated with substance P. Suitable treatment regimens for treatment according to the present invention include daily treatment by aerosol. Other modes of treatment include continual transdermal infusion, intravenous injection, subcutaneous injection, and orally. Suitable formulations of substance P for administration are any which are pharmaceutically acceptable and in which substance P retains its biological activity. Generally, such formulations are substance P dissolved in normal sterile saline.

### EXAMPLES

In experiments performed to assess the effects of short-term (15 minute), low concentration (pM-uM) treatment with aerosolized substance P (SP) on the pulmonary and immune damage resulting from exposure to cigarette smoke (SSCS, 45 min/d, 7 days), it was observed that exposure to cigarette smoke results in pathological changes in the lung as assessed by several methods as well as the formation of DNA micronuclei. In all cases, SP treatment either prevents or reduces the incidence/severity of such pulmonary damage. Further, SP treatment (either prophylactically or therapeutically) of mice in an experimental lung tumor model reduces the incidence of lung tumors and prolongs animal survival. Thus, SP therapy is useful in preventing and/or treating the pathological consequences associated with exposure to cigarette smoke.

Treatment of SSCS-exposed animals with aerosolized SP after exposure to SSCS also protects against this type of environmental toxicant. Simultaneous treatment with SP was also protective, again demonstrating that SP has both prophylactic and therapeutic effects with regard to exposure to cigarette smoke. It is apparent that SP maintains the cellular integrity and function of the lung after SSCS exposure, as measured by an inhibition of epithelia damage and maintenance of normal dynamic lung compliance. Significantly, loss of dynamic lung compliance and damage to the basement membrane in the lung airways correlates to the amount of and length of exposure to cigarette smoke, and with the development of emphysema and induction of malignancy (19, 24). SP treatment also prevents micronuclei induction in cells obtained from SSCS-exposed animals. Such genetic damage correlates with the early stages of carcinogenesis (19, 24). Finally, SP treatment appears to activate the pulmonary immune system as shown by its ability to inhibit lung tumor formation (and restore damaged immune function in previous studies; 7, 10), and its ability to activate lung immune defense mechanisms (i.e., cytokine secretion by PAM). These latter findings may help to explain in part the anti-tumor actions of SP. In fact, SP has been shown in other studies also to activate the innate as well as the adaptive immune systems (15).

### Example 1

### Aerosolized Substance P Attenuates Cigarette Smoke-Induced Cellular Damage in the Lung

C57B1/6 (B6, Jackson Labs) mice were utilized. Mice were used at an age of 8-12 weeks, 25-35 grams in weight. Female animals only were utilized. All animals were housed in the animal facility of the Dept. of Animal Resources at The University of Arizona Health Sciences Center. Animals were used in AAALAC-approved protocols.

Aerosolized exposures were performed using a DeVilbiss Ultra-Neb nebulizer (Model 099HD, Somerset, Pa.). Animals were exposed in a nose-only presentation while held in individual subject loading tubes similarly to that previously described (12). The tubes were nose cone-fitted to receiving adapters that originated from the common exposure chamber (volume 0.0027 m3, IN-TOX, Albuquerque, N. Mex.). Nose-only exposure was employed to minimize ingestion of toxicants during grooming and to more closely simulate occupational exposure. Animals were rotated on a daily basis through the 24 adapter positions on the exposure chamber to minimize proximity to the toxicant source as a variable in exposure. Exposure concentration was determined by a seven-stage cascade impactor (IN-TOX) from changes in plate weights and measured immediately after each exposure. Cascade impactor plates were weighed on an electronic analytical balance (Mettler Instrument Corp, Hightstown, N.J.). Samples were taken from the two heaviest plate deposits and used for gas chromatograph (GC) analysis after each exposure. GC analysis was used for comparison of total plate deposit with simultaneously obtained carbon bead absorption during mock exposure for determination of aerosol to vapor mass ratio (A/V). Through repeated trials the A/V was found to reproducible at a range of 1.2-1.8 (mean+1.5). The vacuum was applied to the exposure chamber of the side opposite the fuel source. Unused animal exposure ports were sealed during the exposure period. Daily measurements of relative humidity, temperature and barometric pressure were made at the time of exposure and, through stepwise regression techniques, excluded as significant causes of variability in toxicant concentration. Previous experiments have demonstrated that exposure concentration during the one-hour exposure period is constant, and that the exposure over the 7-day period varies less than 10% as measured by the SEM of the exposure concentration. Sham exposures (controls) consisted of animals exposed to air only.

Mice were exposed to side-stream cigarette smoke as previously described (23). Briefly, mice were placed in the exposure chamber and exposed for 45 minutes/day for 7 days to side-stream cigarette smoke from 1R4 standard research cigarettes. The cigarette was lit, placed upright in a ring clamp and the smoke was directed into a funnel for distribution through the chamber (at a concentration designed to simulate human exposure in a smokey bar).

The Substance P (SP) agonist, [Sar⁹, Met (O₂)¹¹-substance P] was obtained from Sigma Chemicals (St. Louis, Mo.) and used after reconstitution in sterile saline.

Mice were treated with the SP analog [Sar⁹, Met (O₂)¹¹-substance P] for 15 minutes at the indicated doses following the cigarette smoke exposures as previously described (7, 10). Other than the genetically engineered SP experiments, all SP treatments were by the aerosol route using the previously described exposure chambers.

For each of the tissues and organs wet weights were determined using a microbalance after removal, when possible. Organs were carefully cleaned of surrounding tissue and fat prior to weighing. After processing of the organs into single cell suspensions (using a homogenizer), cell numbers and viabilities were determined by trypan blue dye staining. Cell numbers and viabilities were again determined after centrifugation of the cell suspensions through density gradients to obtain viable mononuclear cells. Only viable cells were used in the functional assays.

The lungs were prepared for morphometric study by injection of half-strength Kamovsky's Fluid into a major artery at a constant pressure of 20 cm H₂O for 1 h at room temperature. The fixed tissue was then tied off at the artery with #4 suture. The Pathology Core Laboratories of the Southwestern Environmental Health Science Center evaluated the fixed tissue for pathological changes. The fixed tissues were sliced, dehydrated and post-fixed in osmium tetroxide, dehydrated and embedded in Epon-Araldite for high-resolution electron microscopy. Thin sections were cut from the embedded tissue with a diamond knife, mounted on 200 um copper mesh grids, and doubly stained with lead citrate and uranyl acetate. The tissue was viewed and photographed using a Phillips CM12 electron microscope (maximum magnification of 19,000×) (21).

Mice were anesthetized with ketamine hydrochloride (80 mg/kg), xylazine (10 mg/kg) and acepromazine maleate (3 mg/kg). A tracheostomy was performed, with the insertion of a Teflon intravenous catheter (20 gauge, Critikon, Tampa Bay, Fla.) serving as an endotracheal tube. The mice were placed under pressure-controlled respiration (Kent Scientific, Litchfield, Conn.) and were given an intraperitoneal injection of gallamine triethiodide (8 mg/kg) to suppress spontaneous breathing. Airflow was measured with a pneumotachograph (Fleisch #0000, Instrumentation Associates, New York, N.Y.) that was coupled to a differential pressure transducer (Validyne, Northridge, Calif.). Airflow and pressure signals were used to measure dynamic lung compliance, total lung compliance and pulmonary resistance. These lung function parameters were measured with a modified PEDS-LAB (Medical Associated Services, Hatfield, Pa.) pulmonary function system. Pulmonary function measurements were normalized to individual animal weight (21).

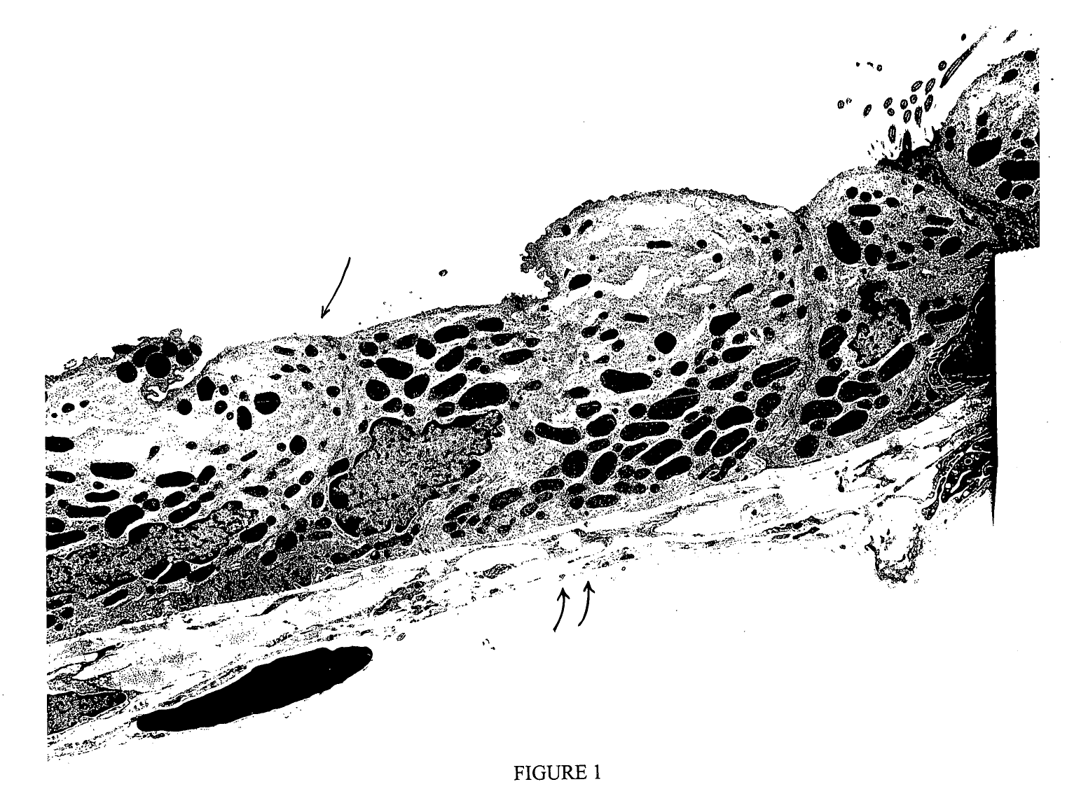

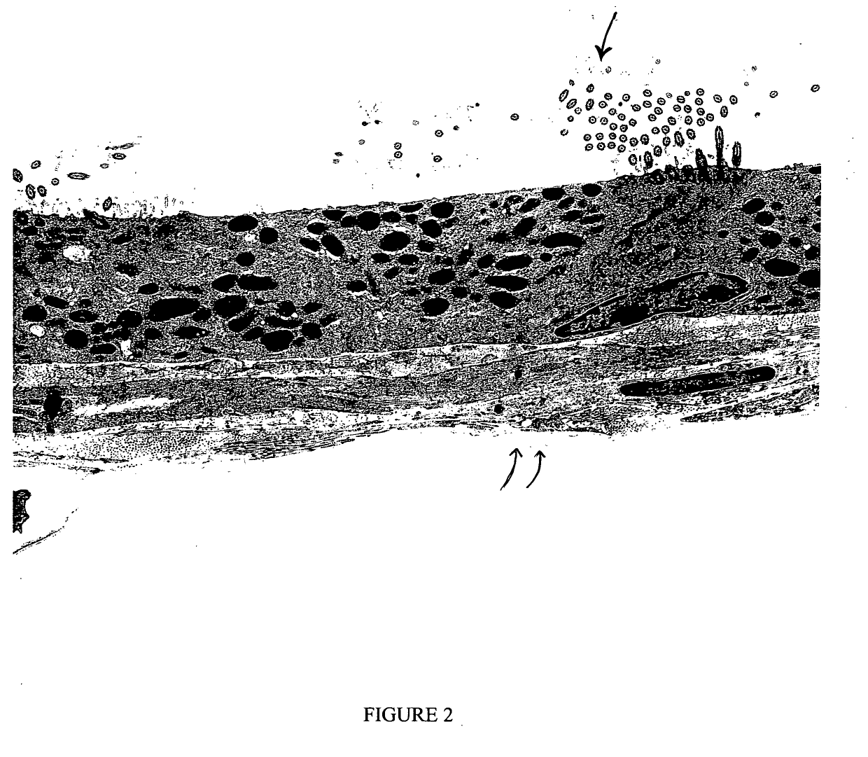

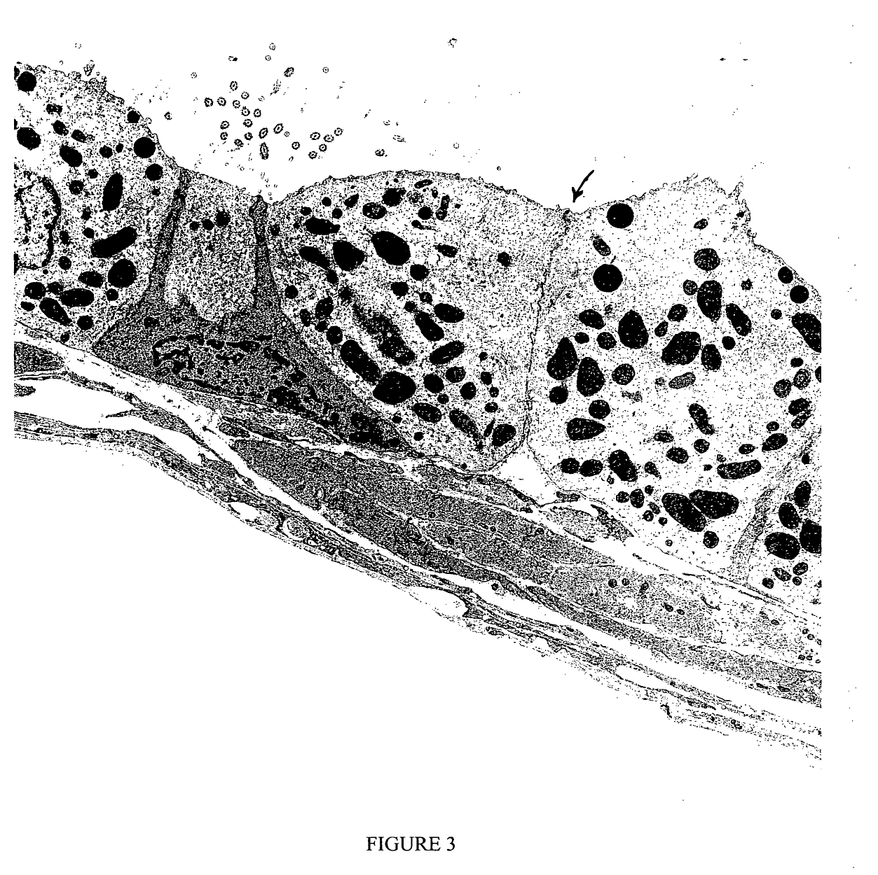

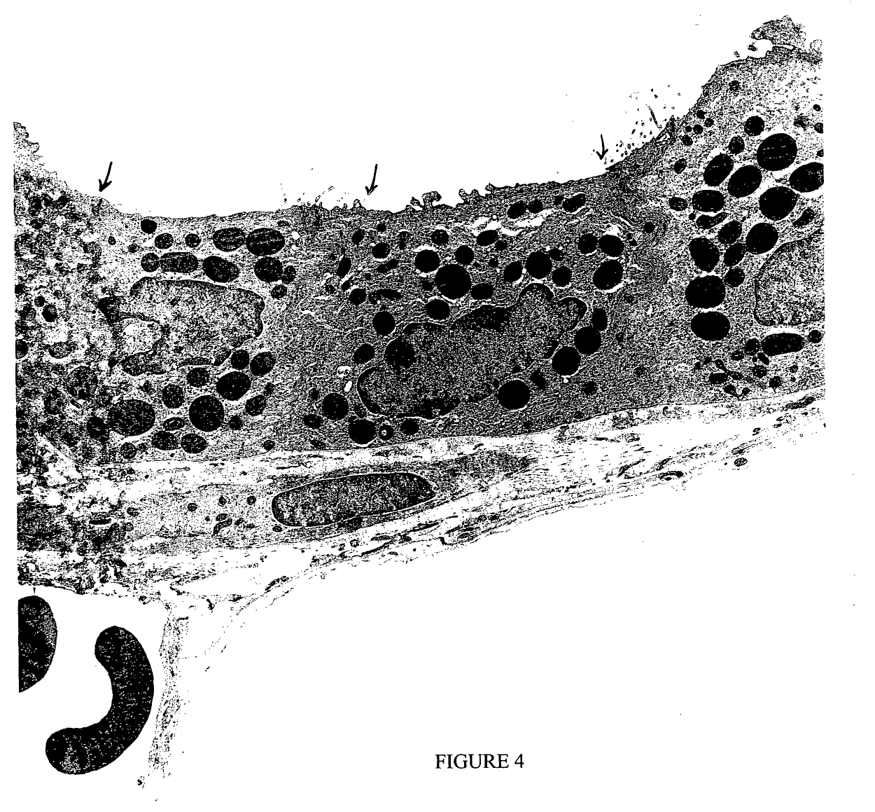

Animals were exposed to SSCS for 45 min/day for 7 days from 1R4 standard research cigarettes (cigarette is lit and then placed upright in a ring clamp and the smoke is directed into a funnel for distribution through our exposure chamber) at a concentration designed to simulate a “smokey bar” scenario for human exposure to sidestream cigarette smoke. The sidestream cigarette smoke is considered highly toxic, even compared to the mainstream cigarette smoke that a human smoker inhales, due to the low combustion temperature of the smoldering cigarette. Subsequent to the exposures, some mice were treated with 1 uM-aerosolized SP for 15 minutes. At the end of one week the animals were sacrificed and lungs removed for electron microscopic analysis. As shown in FIG. 1, exposure to SSCS resulted in basement membrane destruction in the lungs, similar to previous reports (21). Treatment with SP however, attenuated such deterioration of the lung epithelium after SSCS exposure (FIG. 2). The structure of the airway is characterized by airway epithelial cells anchored to a basement membrane. Another basement membrane serves as the anchor for the airway endothelial cells. The SSCS electron micrograph is characterized by swelling in both basement membrane areas of the airway structure and loss of cilia on the surface of the airway epithelium. In FIG. 1 (SSCS), the single arrow shows disruption of the alveolar epithelial cells due to SSCS exposure, while the double arrows indicate swelling in the two basement membrane areas in the airway structure. In FIG. 4 (SSCS), arrows indicate cell swelling and no easily identifiable cell membranes between airway cells. In FIG. 2 (SSCS+SP), the arrows indicate an intact airway epithelium with no swelling present in the basement membranes of the airway structure. In FIG. 3 (SSCS+SP) the arrows indicate identifiable cell membranes between airway cells. These changes in cellular lung composition are reflected in the lung compliance data presented in Table 1. That is, exposure to SSCS resulted in significant alterations in lung compliance, which was prevented/reversed by treatment with SP immediately after the smoke exposures.

TABLE 1
Substance P Prevents Pathological Changes in
Lung Function due to Cigarette Smoke Exposure.

| Expt. | Condition | N | ml · cm H2O⁻¹ · Kg⁻¹ | Significance |
|---|---|---|---|---|
| | Control | 12 | 3.5 (0.7) | |
| Dynamic Lung | SSCS | 15 | 1.4 (0.004) | |
| Compliance | SSCS + SP | 15 | 2.2 (0.02) | p < 0.0018 |

Mice were exposed to SSCS +/− SP for 1 week as described above. At the end of this time animals were anesthetized and dynamic lung compliance was measured as described. Control animals consisted of mice exposed to air and treated with saline.
Data are presented as the mean +/− SEM.
The p value indicates a significant difference from the SSCS group.

### Example 2

### Substance P Therapy Prevents DNA Damage Due to Cigarette Smoke Exposure

Determination of micronuclei formation was made as described by Fenech (3). Briefly, animals were exposed to cigarette smoke ±SP treatment. Viable mononuclear cells were isolated from peripheral blood and bone marrow, stimulated with the mitogen PHA for 44 h, and treated with cytochalasin B for 28 h. Cytocentrifuge preparations were made, cells fixed and then analyzed at 1000× for micronuclei formation. At least 1000 cells were analyzed for each preparation.

Exposure to cigarette smoke results in genetic changes that can cause malignant cellular transformation (21, 22). Animals were exposed to SSCS as described above and sacrificed after 7 days. Viable mononuclear cells were isolated from peripheral blood and bone marrow, stimulated with the mitogen PHA for 44 h, and treated with cytochalasin B for 28 h. Cytocentrifuge preparations were made, cells fixed and then analyzed at 1000× for micronuclei formation. At least 1000 cells were analyzed from each preparation. As shown in Table 2, SSCS exposure resulted in an approximately 10-fold increase in micronuclei formation observed in cells isolated from both the peripheral blood and bone marrow of the exposed animals (as compared to sham-exposed control animals). Micronuclei formation in combination with damage of lung epithelia can result on pathological conditions such as emphysema and cancer (21). Treatment with SP immediately after SSCS exposure resulted in levels of micronuclei formation comparable to control animals, in both blood and bone marrow cells.

TABLE 2
Substance P Inhibits Micronuclei Formation Due to Cigarette Smoke.

Percent Micronuclei

| Group | N | Bone Marrow | Blood |
|---|---|---|---|
| Control | 8 | 0.002 +/− 0.004 | 0.002 +/− 0.004 |
| SSCS | 6 | 0.17 +/− 0.4* | 0.02 +/− 0.01* |
| SSCS + SP | 6 | 0 +/− 0* | 0.01 +/− 0.01* |

Mice were exposed to SSCS +/− SP as described in Table 1. At the end of 7 days the animals were euthanized, and viable cells isolated from the bone marrow and peripheral blood. The incidence of micronuclei formation in a minimum of 1000 cells was evaluated as described. Data are presented as the percentage of cells in the indicated tissue that displayed micronuclei.
Data are presented as the mean +/− SD.
*p < 0.05 as compared to the Control group.

### Example 3

### Substance P Treatment Activates Lung Immune Mechanisms and Inhibits Tumor Incidence

Damage of lung epithelia in combination with the induction of micronuclei formation can result in pathological conditions such as emphysema and cancer (21). Studies were performed using an experimental tumor model to examine the effects of SP on the development of lung cancer.

Rat pulmonary alveolar macrophages (PAM) were isolated from pathogen-free male Fischer 344 rats (Harlan, Indianapolis, Ind.). The rats were anesthetized intramuscularly with ketamine HCL (80 mg/kg; Parke-Davis, Morris Plains, N.J.), xylazine (10 mg/kg; Mobay Corp., Shawnee, Kans.) and acepromazine maleate (3 mg/kg; Fermenta Animal Health Co., Kansas City, Mo.). A tracheostomy was performed, with the insertion of a Teflon #18 gauge catheter (Critikon, Tampa Bay, Fla.) as an endotracheal tube. The rats were killed by exsanguination of the abdominal aorta. The lungs were removed and lavaged with 3 ml aliquots of normal sterile saline warmed to 37 C for a total of 6 washes. The lavaged total cell numbers and PAM differentials were determined from a 0.2 ml sample by hemocytometer counting and cytocentrifuge preparation stained with Diff-Quik (Dade Diagnostics, Aguada, Puerto Rico), respectively. The remaining lavaged fluid was pooled and centrifuged at 400×g for 10 minutes to obtain a cell pellet. The saline supernatant was decanted and cells were resuspended in BRFF-RluE media supplemented with penicillin/streptomycin. Cells were then counted using a standard hemocytometer and placed in 12 well plates at a density of 10⁴ cells/ml. After 1 h of adherence at 37 C, cells were washed once with media to remove non-adherent cells and cultured with fresh media. These cultured cells were used as a source of PAM (25).

TNF-alpha secretion by pulmonary alveolar macrophages (PAM) was measured by ELISA (R&D Systems, Minneapolis, Minn.) according to the manufacturer's instructions (25).

B16 tumor cells (H-2^{b}) were obtained from the American Type Cell Collection (ATCC) and grown in DMEM media (Sigma, St. Louis, Mo.) supplemented with 10% fetal bovine serum (Hyclone, Colo.), antibiotics, nonessential amino acids and glutamine as. Cells in log growth were used for all experiments. An experimental lung metastasis model was utilized in which B16 tumor cells (0.5×10⁶ cells in 100 ul saline) were injected intravenously into syngeneic B6 mice. At 7-10 days post-injection the animals were sacrificed and the visually prominent (black) tumors on the surface of both lungs were enumerated.

The Student's t-test was utilized to analyze the data presented. A p value of 0.05 (or lower) was used as a minimally significant difference.

As shown in Table 3, intravenous injection of syngeneic tumor cells resulted in large numbers of lung tumor colonies (30-242) after a short period to time (7-10 days). However, treatment of the animals with aerosolized SP either at the time of (Experiment 1) or after (Experiments 2 & 3) tumor induction resulted in significant inhibition of lung tumor formation (58-97%). Further, SP therapy also resulted in prolongation of animal survival (Experiment 1; an increase of 24%).

### Example 4

### Endogenous Substance P Secretion

The cDNA sequence encoding mature substance P (SP) was acquired from Gen-Bank, while the Ig-kappa chain leader sequence with a kozak sequence was taken from the plasmid pSecTag2B (Invitrogen, San Diego, Calif.). A new gene construct was designed to encode a secreted type of SP by adding the Ig-kappa leader sequence before the SP sequence, with a Nhe I site at the 5′ end and a Not I site at the 3′ end. This construct was cloned into the Nhe I/Not I site of the pCI-neo plasmid (Promega, Madison, Wis.). B16 tumor cells were transfected by standard methods with the gene construct and stable transfectants selected for the in vivo experiments. Secreted SP was measured by ELISA (Cayman, Ann Arbor, Mich.) according to the manufacturer's instructions. SP gene expression was also confirmed by the reverse transcription polymerase chain reaction (RT-PCR), using kits purchased from Stratagene, Inc (LaJolla, Calif.).

Interestingly, genetic modification of tumor cells to endogenously secrete SP at low levels (75 pg/ml; Table 3 Experiment 4) reduced the number of lung tumor colonies formed upon administration to mice. This observation may imply immune system activation (4, 7). It should be noted that B16 tumors cells do not secrete any detectable SP normally. Consistent with this implication was the finding that incubation of lung macrophages (PAM; Table 4) with either nM or uM concentrations of SP in vitro resulted in cellular activation as measured by secretion of the cytokine, TNF-alpha.

TABLE 3
Substance P Therapy Reduces Experimental
Lung Cancer Colony Formation.

| | | #Tumors/ | | Days |
|---|---|---|---|---|
| | N | 2 Lungs | % Change | Survival/Other |
| Expt. 1 | | | | |
| B16 | 4 | 105 +/− 12 | | 25 +/− 3 days |
| B16 + SP | 4 | 39 +/− 4* | −63% | 31 +−/4 days |
| | | | | (+24%)* |
| Expt. 2 | | | | |
| B16 | 4 | 30 +/− 5 | | |
| B16 + SP | 4 | 1 +/− 0.3* | −97% | |
| Expt. 3 | | | | |
| B16 | 7 | 242 +/− 54 | | |
| B16/SP | 8 | 102 +/− 32* | −58% | |
| Expt. 4 | | | | |
| B16 | 8 | 190 +/− 33 | | |
| B16/SP | 8 | 78 +/− 30* | −59% | |
| | | | (75 pg/ml secreted | |
| | | | SP/10⁶ cells/24 h) | |
| Mice were injected intravenously with syngeneic B16 tumor cells to simulate lung cancer as described above. At the end of 7-14 days the animals were sacrificed (except in Experiment 1 in which a separate group of mice were maintained to evaluate survival), and lung colonies in both lungs enumerated. In Experiment 1, aerosolized SP was given at the time of tumor induction, while in Experiments 2 & 3, aerosolized SP was administered at day 7 following tumor induction. Experiment 4 | | | | |
| # shows the results obtained using genetically modified tumor cells (see above for details). | | | | |

Data are presented as the mean +/− SD.
*p < 0.05 as compared to B16 alone.

TABLE 4
Activation of Pulmonary Macrophages By Substance P Addition.

| Cells | Addition | N | Fold-Increase TNFa | Significance |
|---|---|---|---|---|
| PAM | 8 nM SP | 5 | 3.2X | p < 0.05 |
| PAM | 1 uM SP | 5 | 5.4X | p < 0.05 |

PAM were isolated as described in Materials and Methods and incubated overnight in the presence of the indicated concentration of SP. TNF-alpha secretion into the culture media was assessed by ELISA. Baseline level of cytokine secretion by unstimulated PAM was 8.3 pg/ml per 1 × 10⁶ cells.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### REFERENCES

1. Wang, S. and Witten, M. L. Environmental tobacco smoke and lung function. In: Environmental Tobacco Smoke, Eds. Watson and Witten, CRC Press, New York, N.Y., 2001, p. 301-307.
2. Chen, H., Witten, M. L., Pfaff, J. K., Lantz, R. C., and Carter, D. JP-8 jet fuel exposure increases alveolar permeability in rats. FASEB J. 1992, 6:A1064.
3. Harris, D. T., Sakiestewa, D., Robledo, R. and Witten, M. Immunotoxicological effects of exposure to JP-8 jet fuel. Toxicology and Industrial Health 1997, 13(1):43-56.
4. Harris, D. T., Sakiestewa, D., Robledo, R. and Witten, M. Protection from JP-8 Jet Fuel Induced Immunotoxicity by Administration of Aerosolized Substance P. Toxicology and Industrial Health, 1997, 13(5): 571-588.
5. Harris, D. T., Sakiestewa, D., Robledo, R. and Witten, M. Short-Term Exposure to JP-8 Jet Fuel Results in Longterm Immunotoxicity. Toxicology and Industrial Health, 1997, 13(5): 559-570.
6. Harris, D T, Sakiestewa, D., Robledo, R. F., Young, R. Scott and Witten, M. Effects of short term JP-8 jet fuel exposure on cell-mediated inmnunity. Tox. & Indus. Health 2000, 16:78-84.
7. Harris, D T, Sakiestewa, D., Titone, D., Robledo, R. F., Young, R. S. and Witten, M. Substance P as prophylaxis for JP-8 jet fuel-induced immunotoxicity. Tox. & Indus. Health 2000, 16:253-259.
8. Harris, D T, Sakiestewa, D., Titone, D., Robledo, R. F., Young, R. S. and Witten, M. Jet fuel induced immunotoxicity. Tox. & Indus. Health 2000, 16:261-265.
9. Pfaff, J., Parliman, G., Parton, K., Lantz, R., Chen, H., Hays, A., and Witten, M. Pathologic changes after JP-8 jet fuel inhalation in Fischer 344 rats. FASEB J. 1993, 7:A408.
10. Pfaff, J., Parton, K., Lantz, R. C., Chen, H., Hays, A., and Witten, M. L. Inhalation exposure to JP-8 jet fuel alters pulmonary function and substance P levels in Fischer 344 rats. J. Appl. Toxicol. 1995, 15(4):249-256.
11. Witten, M. L., Leeman, S. E., Lantz, R. C., Joseph, P. M., Burke, C.H., Jung, W. K., Quinn, D. and Hales, C A. Chronic jet fuel exposure increases lung substance P (SP) concentration in rabbits. In: Substance P and Related Peptides : Cellular and Molecular Physiology (S. E. Leeman, ed). International Symposium, University of Massachusetts and the New York Academy of Science, New York, 1990, p. 29.
12. Witten, M. L., Pfaff, J., Lantz, R. C., Parton, K. H., Chen, H., Hays, A., and Leeman, S. E. Capsaicin pretreatment before JP-8 jet fuel exposure causes a large increase in airway sensitivity to histamine in rats. Regul. Pept. 1992, S1: S176.
13. Pfaff, J., Parton, K., Lantz, R. C., Chen, H., Hays, A., and Witten, M. L. Inhalation exposure to JP-8 jet fuel alters pulmonary function and substance P levels in Fischer 344 rats. J. Appl. Toxicol. 1995, 15(4):249-256.
14. Witten, M. L., Pfaff, J., Lantz, R. C., Parton, K. H., Chen, H., Hays, A., and Leeman, S. E. Capsaicin pretreatment before JP-8 jet fuel exposure causes a large increase in airway sensitivity to histamine in rats. Regul. Pept. 1992, S1: S176.
15. DeSanctis, G. T., App, E. M., Trask, J. K., DeSanctis, B. I., Remmers, J. E., Green, F. H. Y., Man, S. F. P. and King, M. Resorptive clearance and transepithelial potential difference in capsaicin-treated F344 rats. J. Appl. Physiol. 1990, 68(5): 1826-1832.
16. Lundberg, J. M., Hokfelt, T., Martling, C. R., Saria, A., and Cuello, C. Substance P-immunoreactive sensory nerves in the lower respiratory tract of various mammals including man. Cell Tissue Res. 1984, 235:251-261.
17. Lundberg, J. M., Lundblad, L., Anggard, A., Martling, C. R., Thoedorsson-Norheim, E., Stjarne, P. and Hokfelt, T. G. Bioactive peptides in capsaicin-sensitive C-fiber afferents of the ariways. In: Kaliner, M. A. & P. J. Barnes (Eds), The airways: neural control in health and disease, Marcel Dekker (New York) 1987, p. 417-445.
18. Martling, C. R., Theodorsson-Norheim, E. and Lundberg, J. M. Occurrence and effects of multiple tachykinins; substance P, neurokinin and neuropeptide K in human lower airways. Life Sci. 1987, 40:1633-1643.
19. Fuller, R. W., Maxwell, D. L., Dixon, C. M. S., McGregor, G. P., Barnes, V. F., Bloom, S. R., and Barnes, P. J. Effect of substance P on cardiovascular and respiratory function in subjects. J. Appl. Physiol. 1987, 62:1473-1479.
20. Robledo, R. F. and Witten, M. L. Substance P attenuates lung injury caused by hydrocarbon exposure. In: Proceedings of the 1995 Tachykinins International Meeting, Florence, Italy, 1995, p. 190.
21. Wang, S. and Witten, M. L. Environmental tobacco smoke and lung function. In: Environmental Tobacco Smoke, Eds. Watson and Witten, CRC Press, New York, N.Y., 2001, p. 301-307.
22. Pimm, P., Shephard, R. J. and Silverman, F. Physiological effects of acute exposure to cigarette smoke. Arch. Environ. Health 1978, 33:201.

1. A method of ameliorating or preventing damage caused by cigarette smoke, comprising:
administering substance P or a bioactive analog thereof via aerosol inhalation to a subject who has or will be exposed to cigarette smoke, wherein the bioactive analog is selected from the group consisting of [Met-OH11]-substance P, [Met-OMe11]-substance P, [Nle11]-substance P, [Pro9]-substance P, [Sar9]-substance P, [Tyr8]-substance P, [p-Cl-Phe7,8]-substance P, and [Sar9,Met (02)11]-substance P.

2. The method of claim 1 wherein the subject has been exposed to cigarette smoke.

3. The method of claim 1 wherein the subject has been exposed to side-stream cigarette smoke.

4. The method of claim 1 wherein the subject is exposed to cigarette smoke subsequent to substance P aerosol inhalation.

5. The method of claim 1 wherein the substance P is administered at 0.1 to 10 μM.

6. The method of claim 1 wherein the substance P is administered at 0.5 to 5 μM.

7. The method of claim 4 wherein the cigarette smoke is side-stream cigarette smoke.

8. The method of claim 1 wherein the cigarette smoke is main-stream cigarette smoke.

9. The method of claim 1 wherein the substance P is administered in an amount sufficient to prevent micronuclei formation in the subject's bone marrow cells.

10. The method of claim 1 wherein the substance P is administered in an amount sufficient to prevent micronuclei formation in the subject's blood cells.

11. The method of claim 1 wherein the substance P is administered in an amount sufficient to increase dynamic lung compliance.

12. The method of claim 1 wherein the substance P is administered in an amount sufficient to prevent damage or remedy damage to basement membrane of endothelial cells of airways.

13. A method of ameliorating or preventing damage caused by cigarette smoke, comprising:
administering substance P or a bioactive analog thereof via a filter of a cigarette, cigar or other smoking product to a subject, wherein the bioactive analog is selected from the group consisting of [Met-OH11]-substance P, [Met-OMe11]-substance P, [Nle11]-substance P, [Pro9]-substance P, [Sar9]-substance P, [Tyr8]-substance P, [p-Cl-Phe7,8]-substance P, and [Sar9,Met (02)11]-substance P.

14. The method of claim 13 wherein the substance P is administered in an amount sufficient to prevent micronuclei formation in the subject's bone marrow cells.

15. The method of claim 13 wherein the substance P is administered in an amount sufficient to prevent micronuclei formation in the subject's blood cells.

16. The method of claim 13 wherein the substance P is administered in an amount sufficient to increase dynamic lung compliance.

17. The method of claim 13 wherein the substance P is administered in an amount sufficient to prevent damage or remedy damage to basement membrane of endothelial cells of airways.

18. A method of ameliorating or preventing damage caused by cigarette smoke, comprising:
administering substance P or a bioactive analog thereof via a gum or lozenge to a subject who has or will be exposed to cigarette smoke, wherein the bioactive analog is selected from the group consisting of [Met-OH11]-substance P, [Met-OMe11]-substance P, [Nle11]-substance P, [Pro9]-substance P, [Sar9]-substance P, [Tyr8]-substance P, [p-Cl-Phe7,8]-substance P, and [Sar9,Met (02)11]-substance P.

19. The method of claim 18 wherein the subject has been exposed to cigarette smoke.

20. The method of claim 18 wherein the subject has been exposed to side-stream cigarette smoke.

21. The method of claim 18 wherein the subject is exposed to cigarette smoke subsequent to administration of the gum or lozenge.

22. The method of claim 18 wherein the substance P is at a concentration of 0.1 to 10 μM.

23. The method of claim 18 wherein the substance P is at a concentration of 0.5 to 5 μM.

24. The method of claim 18 wherein the cigarette smoke is side-stream cigarette smoke.

25. The method of claim 18 wherein the cigarette smoke is main-stream cigarette smoke.

26. The method of claim 18 wherein the substance P is administered in an amount sufficient to prevent micronuclei formation in the subject's bone marrow cells.

27. The method of claim 18 wherein the substance P is administered in an amount sufficient to prevent micronuclei formation in the subject's blood cells.

28. The method of claim 18 wherein the substance P is administered in an amount sufficient to increase dynamic lung compliance.

29. The method of claim 18 wherein the substance P is administered in an amount sufficient to prevent damage or remedy damage to basement membrane of endothelial cells of airways.

30. A cigarette filter comprising substance P or a bioactive analog thereof.

31. A gum or lozenge comprising substance P or a bioactive analog thereof.

32. A method of ameliorating or preventing damage caused by cigarette smoke, comprising:
administering a polynucleotide to a human or animal, said polynucleotide encoding a secretable substance P protein or a bioactive analog wherein the bioactive analog is selected from the group consisting of [Met-OH11]-substance P, [Met-OMe11]-substance P, [Nle11]-substance P, [Pro9]-substance P, [Sar9]-substance P, [Tyr8]-substance P, [p-Cl-Phe7,8]-substance P, and [Sar9,Met (02)11]-substance P.

33. The method of claim 32 wherein the subject has been exposed to cigarette smoke.

34. The method of claim 32 wherein the subject has been exposed to side-stream cigarette smoke.

35. The method of claim 32 wherein the substance P is secreted in an amount sufficient to prevent micronuclei formation in the subject's bone marrow cells.

36. The method of claim 32 wherein the substance P is secreted in an amount sufficient to prevent micronuclei formation in the subject's blood cells.

37. The method of claim 32 wherein the substance P is secreted in an amount sufficient to increase dynamic lung compliance.

38. The method of claim 32 wherein the polynucleotide is administered via a viral vector.

39. The method of claim 32 wherein the polynucleotide is administered via the nose.

40. The method of claim 32 wherein the polynucleotide is administered via inhalation.

41. The method of claim 32 wherein the polynucleotide is administered to the lungs.


SEQUENCE LISTING (1 sequence)

SEQ ID NO: 1 - protein, 11 aa, Homo sapiens
RPKPQQFFGLM